(19) United States
(12) Patent Application Publication
(10) Pub. No.: US 2021/0078003 A1
(43) Pub. Date: Mar. 18, 2021

(54) SEQUENCING FLOWCELLS

(71) Applicants: Qiagen GmbH, Hilden (DE); Qiagen Sciences, LLC, Germantown, MD (US)

(72) Inventors: Sebastian Koeber, Leichlingen (DE); Maximilian Focke, Duesseldorf (DE); Luisa Andruzzi, Concord, MA (US)

(73) Assignees: Qiagen GmbH, Hilden (DE); Qiagen Sciences, LLC, Germantown, MD (US)

(21) Appl. No.: 16/963,690

(22) PCT Filed: Jan. 25, 2019
(86) PCT No.: PCT/US2019/015089

§ 371 Date: Jul. 21, 2020

Related U.S. Application Data
(60) Provisional application No. 62/622,215, filed on Jan. 26, 2018.

(51) Int. Cl. B01L 3/00; C12Q 1/6869

(52) U.S. Cl. CPC B01L 3/502761; C12Q 1/6869; B01L 2300/021; B01L 2300/168; B01L 2300/0816; B01L 2300/0851; B01L 2300/0887; B01L 2300/0636

(57) ABSTRACT

A flowcell device for a sequencing by synthesis instrument. The flowcell device has a fluid inlet configured to receive one or more liquid reagents, a fluid outlet configured to pass the one or more liquid reagents, and a channel extending between and fluidly connecting the fluid inlet and the fluid outlet. At least a portion of the channel comprises a reflective structure configured to retain a plurality of sequencing targets thereon. The reflective structure includes at least a metal oxide layer and a film having a first surface and a second surface opposed the first surface. The first surface of the film is disposed on the metal oxide layer and the second surface of the film is configured to receive a plurality of sequencing targets immobilized thereon.

### CROSS REFERENCE TO RELATED APPLICATION

This application is related to, and claims the benefit of priority of, U.S. Provisional Application No. 62/622,215, entitled SEQUENCING FLOWCELLS, filed on 26 Jan. 2018, the contents of which are incorporated herein by reference in their entirety for all purposes.

### FIELD OF INVENTION

The present invention relates generally to flowcell devices for sequencing by synthesis instruments and methods of manufacturing the same.

### BACKGROUND OF THE INVENTION

DNA sequencing instruments are typically used to determine DNA molecular sequences. Such instruments are useful for clinical studies, diagnostics, so-called “personalized medicine” (medical treatment tailored to an individual's genetic content or the like), and so on. Current instruments for performing DNA sequencing use a variety of technologies to analyze the base pairs that form the DNA sequence. For example, some instruments perform sequencing on single-stranded DNA molecule fragments (DNA templates) that are fixed in place inside a flowcell. The flowcell is essentially a small chamber in which the DNA templates are subjected to a series of nucleobase extension processes. Each successive extension is detected to determine the base pair sequence of each DNA template. The flowcell provides an environment to hold the DNA templates during the extension process, and also during the inspection process to read each extended base pair.

Many sequencing-by-synthesis instruments use an optical system such as a microscope to detect the nucleobase extensions, although non-optical systems are also known. A typical optical instrument uses visible chemical labels to determine the identity of each extended base pair. For example, each nucleobase that makes up the DNA molecule (adenine, guanine, cytosine and thymine) may be labeled with a unique fluorescent probe that is visible through the microscope. The label is read each time the DNA template is extended, and then the label is removed to make way for the next base pair extension.

### SUMMARY OF THE INVENTION

According to aspects of the present invention, provided are flowcell devices for sequencing by synthesis instruments and, particularly, flowcell sequencing devices having certain characteristics and methods of manufacturing the same.

In one exemplary aspect, a flowcell device is provided for a sequencing by synthesis instrument. The flowcell device includes a fluid inlet configured to receive one or more liquid reagents; a fluid outlet configured to pass the one or more liquid reagents; and a channel extending between and fluidly connecting the fluid inlet and the fluid outlet. At least a portion of the channel comprises a reflective structure configured to retain a plurality of sequencing targets thereon. The reflective structure has a metal oxide layer and a film having a first surface and a second surface opposed the first surface. The first surface of the film is disposed on the metal oxide layer and the second surface of the film is configured to receive a plurality of sequencing targets immobilized thereon.

In another exemplary aspect, a method is provided for manufacturing flowcell devices configured for sequencing by synthesis. The method for manufacturing such flowcell devices includes forming a reflective structure comprising at least two layers by binding a metal oxide layer to a film. The film has a first surface bonded to the metal oxide layer and a second surface that is opposed the first surface. The second surface of the film is configured to receive a plurality of sequencing targets immobilized thereon. The method further includes coupling an adhesive layer to a flowcell plate comprising a rigid material. The adhesive layer has a passageway extending therethrough. Additionally, the method includes forming a flowcell device including a fluid inlet, a fluid outlet, and a channel extending between and fluidly connecting the fluid inlet and the fluid outlet by coupling the reflective structure to the adhesive layer to form the channel from the passageway between the second surface of the film of the reflective structure and the flowcell plate.

### BRIEF DESCRIPTION OF THE DRAWINGS

A better understanding of the exemplary embodiments may be understood by reference to the attached drawings, in which like reference numbers designate like parts. The drawings are exemplary and not intended to limit the claims in any way.

FIG. 1 is an exploded view of a first embodiment of a flowcell device in accordance with aspects of the present invention.

FIG. 2 is a cross-sectional view of the reflective structure of FIG. 1 with sequencing targets immobilized thereon.

FIG. 3 is an exploded view of a second embodiment of a flowcell sequencing device according to aspects of the present invention.

FIG. 4A is top view of an embodiment of a reflective structure having a penultimate layer comprising niobium oxide.

FIG. 4B is top view of an embodiment of a reflective structure having a penultimate layer comprising silicon oxide.

FIG. 5 is a microscope image of a reflective structure having a penultimate layer comprising of silicon oxide that suffered significant loss of the sequencing targets.

FIG. 6 is a heat map indicating the loss of sequencing beads for a reflective structure having a penultimate layer comprising niobium oxide and a reflective structure having a penultimate layer comprising silicon oxide.

FIG. 7 depicts an embodiment of a method for manufacturing a flowcell device for sequencing by synthesis instruments according to aspects of the invention.

FIG. 8 is a schematic depicting metal oxide layers disposed on a silicon oxide layer by atomic layer deposition.

FIG. 9A is a graph illustrating a simulated plot of the reflection for a glass coated with various thicknesses of niobium oxide.

FIG. 9B is a schematic of the glass coated with various thicknesses of niobium oxide associated with FIG. 9A.

### DETAILED DESCRIPTION

Sequencing by synthesis (hereafter “SBS”) techniques typically use various multi-component mixtures during each step of the SBS process, such as during single-base extension, imaging, terminator/dye removal, and washing steps. Moreover, each of the SBS steps may have different chemical properties due to, e.g., the required chemical make-up of the base buffer for functional molecules, nucleotides, etc.

It has been found that, in some instances, the various processes and chemicals used in SBS steps can place significant demands on the construction of the flowcell in which such processes are performed. The inventors have recognized the need to provide more robust flowcell devices, which have improved compatibility characteristics, for sequencing by synthesis instruments. Descriptions of particular exemplary embodiments follow, but it will be appreciated that the scope of the invention is not limited to any particular example, and the examples may be combined and modified in various ways, as will be understood by one of ordinary skill in the art in view of the present disclosure.

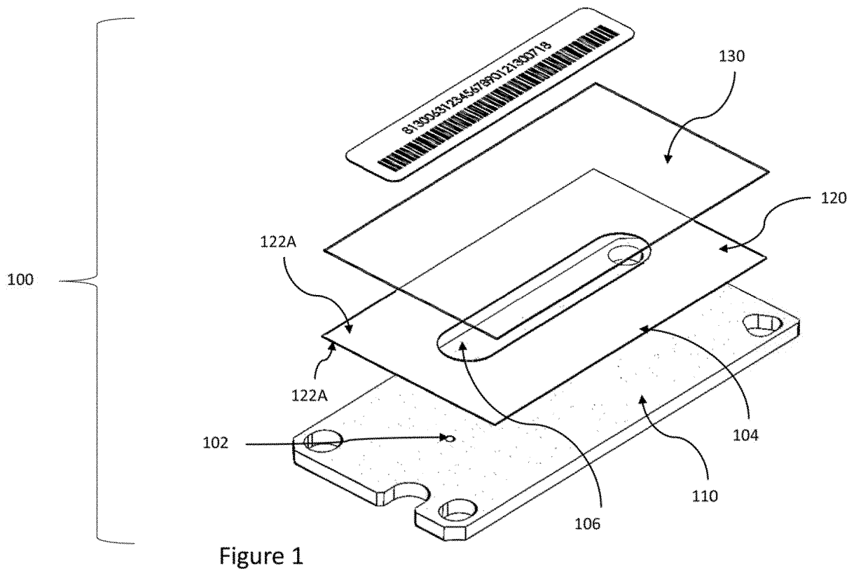

FIG. 1 is an exploded view of a first exemplary embodiment of a flowcell device 100. As a general overview, flow device 100 includes a flowcell plate 110, an adhesive layer 120, and a reflective structure 130.

Flowcell plate 110 may comprise a rigid material such as, metal (e.g., titanium compositions), glass (e.g., borosilicate glass or the like), plastic (e.g., polycarbonate or the like), ceramic compositions, or other suitable materials. As illustrated in FIG. 1, flowcell plate 110 may have a first aperture defining a fluid inlet 102 configured to receive one or more liquid reagents and a second aperture defining a fluid outlet 104 configured to pass the one of more liquid reagents. Flowcell plate 110 may be optically transparent in a range of wavelengths, e.g., to facilitate optical reading processes. Flowcell plate 110 may also have autofluorescence properties. Suitable examples of flowcell devices and flowcell plates are further described in U.S. patent application Ser. Nos. 15/386,490, 12/405,779, and 13/832,509, which are incorporated by reference herein in their entirety for all purposes.

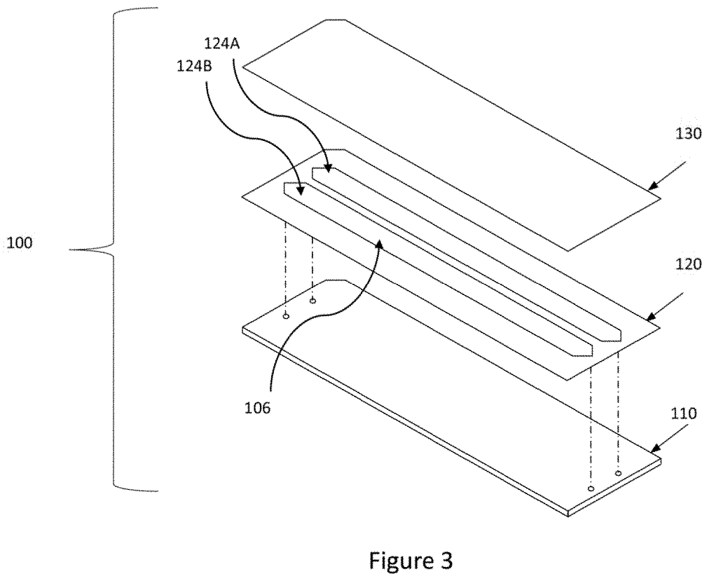

Adhesive layer 120 has at least one surface adapted for attachment. Preferably, adhesive layer 120 has a first surface 122A and an opposed second surface 122B, which are both adapted to attach to a substrate (e.g., flowcell plate 110 and/or reflective structure 130). Adhesive layer 120 may have an adhesive material (e.g., epoxy, glue, etc.) disposed on first and/or second surface 122, which facilitates adhesion of adhesive layer 120 to a particular material, such as a metal, glass, polymer, ceramic, etc. Adhesive layer 120 defines a passageway 124 extending therethrough, e.g., from first surface 122A to second surface 122B of adhesive layer 120. As illustrated in FIG. 3, adhesive layer 120 may define more than one passageways, such as the two passageways 124A and 124b illustrated in the embodiment shown in FIG. 3. The adhesive layer 120 may be made entirely of an adhesive composition or it may comprise a sheet of material with adhesive provided on both sides.

As shown by the embodiment illustrated in FIG. 1, adhesive layer 120 is disposed between flowcell plate 110 and reflective structure 130 (further discussed below), such that passageway 124 forms a channel 106 between the second surface 142B of the reflective structure 130 and flowcell plate 110. Channel 106 extends between and fluidly connects fluid inlet 102 and the fluid outlet 104. In the shown embodiments, the thickness of the adhesive layer defines the height of the flowcell passageway 124. In other cases, the flowcell plate 110 and/or reflective structure 130 may be formed with channels or the like to further define the height of the flowcell passageway 124.

At least a portion of channel 106 faces the reflective structure 130, which is configured to retain a plurality of sequencing targets 144 thereon. In one embodiment, the plurality of sequencing targets 144 on film 140 are in fluid connection with channel 106. In another embodiment, the plurality of sequencing targets 144 on film 140 are positioned within channel 106.

Reflective structure 130 preferably is configured to facilitate inspection of the SBS processes occurring among the sequencing targets 144, and may have optical transmission and reflectance properties to allow such inspection. For example, reflective structure 130 may be formed of two or more layers, such as glass, a metal oxide layer 134 and a film 140. Reflective structure 130 may be configured to reflect near-infrared light waves. Additionally or alternatively, reflective structure 130 may be transparent in the spectral range of 400-700 nm. For example, metal oxide layer 134 may be a translucent layer having a reflectivity selected for proper optical reading with an optical instrument. Metal oxide layer 134 may be formed from one or more metals selected from the group consisting of niobium oxide, zirconium oxide, titanium oxide, and hafnium oxide. In one embodiment, metal oxide layer 134 comprises niobium oxide. In another embodiment, metal oxide layer 134 comprises zirconium oxide. In a further embodiment, metal oxide layer 134 comprises titanium oxide. In yet another embodiment, metal oxide layer 134 comprises hafnium oxide. Because metal oxide layer 134 may be coupled directly to first surface 142A of film 140, metal oxide layer 134 may be the penultimate layer of reflective structure 130. In one embodiment, however, a barrier layer is disposed between metal oxide layer 134 and film 140, as further discussed below.

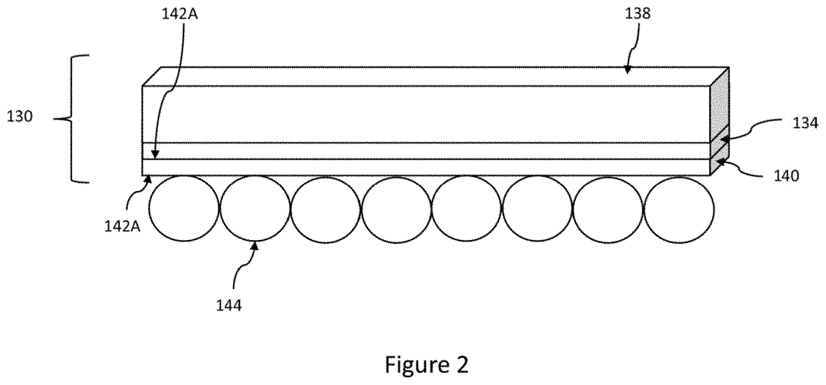

Reflective structure 130 may include a cover layer 138 disposed on a surface of optical mirror structure 136. Cover layer 138 may be configured to protect the metal oxide layer 134 and/or film 140, e.g., by being sized and formed of a material (e.g., borosilicate or other glass compositions) that is resistant to chemicals and/or physical force or pressure, such as scratching. Although the embodiment illustrated in FIG. 2 includes cover layer 138, in another embodiment, cover layer 138 is absent from reflective structure 130.

Reflective structure 130 includes a film 140 having a first surface 142A disposed on metal oxide layer 134 and a second surface 142B that is opposed to first surface 142A and configured to receive a plurality of sequencing targets 144 thereon. Preferably, reflective structure 130 is arranged such that film 140 is the terminal layer reflective structure 130. As illustrated in FIG. 2, one embodiment of the present invention includes a reflective structure 130 arranged such that the penultimate layer of reflective structure 130 is metal oxide layer 134.

Film 140 may comprise a flexible thin film material that is attached to metal oxide layer 134 and/or adhesive layer 120 along a perimeter region, e.g., that surrounds fluid inlet 112 and/or fluid outlet 114, such that the reagents passing through flowcell device 100 exit solely through fluid outlet 114. In one embodiment, the entirety of first surface 142A of film 140 is disposed on metal oxide layer 134. Film 140 provides a biofunctional coating configured to functionalize reflective structure 130 for linking the plurality of sequencing targets 144. For example, film 140 may be functionalized for binding to macromolecules, nucleotides, cells, etc. A suitable film 140 for reflective structure 130 may be AZIGRIP4, which is available from Optics Balzers AG from Liechtenstein. Additional layers may be employed with reflective structure 130, such as the film layers described in U.S. patent application Ser. No. 15/386,490, which is incorporated by reference herein in its entirety for all purposes.

Preferably, film 140 is adapted to retain the plurality of sequencing targets 144 such that the sequencing targets 144 are immobilized on surface 142B of film 140. The plurality of sequencing targets 144 may be retained in an arranged and/or ordered pattern or randomly arranged on second surface 142B of film 140. A coating or treatment may be applied to film 140 to form a scaffold on second surface 142B of film 140 for receiving the plurality of sequencing targets 144 and/or for growing nucleotide molecules. The scaffold may be patterned to minimize overlap of nucleotides forming on the plurality of sequencing targets 144. For example, a hexagonal scaffold pattern may be thermoformed into the film 140 to provide physical locations or film 140 may be treated with a pattern of chemical bonding sites to immobilize the plurality of sequencing targets 144. Additionally or alternatively, film 140 may be treated by structural manipulation, such as by forming wells using embossing techniques or by adding a grid-like layer, to assist with positioning or immobilizing DNA template colonies or to provide other benefits. Film 140 may also be coupled to a polymeric barrier layer disposed between film 140 and metal oxide layer 134. The barrier layer may be configured to protect a layer of reflective structure 130, e.g., metal oxide layer 134, from fluid reagents flowing through the flowcell device when the film is porous. Preferably, the barrier layer is formed of a material comprising poly(methyl methacrylate), cyclo olefin polymers, cyclic olefin copolymer, polycarbonate and/or the like. Other alternatives will be apparent to persons of ordinary skill in the art in view of the present disclosure.

The plurality of sequencing targets 144 are adapted to receive and/or retain nucleotides, functional molecules, and the like. For example, the plurality of sequencing targets 144 may be configured to hold a DNA and/or RNA template colony comprising a plurality of clonal DNA template strands. The specific details of the chemical reactions are not relevant to the present disclosure, and are not described herein. However, examples of sequencing processes are described in U.S. Patent Application Publication Nos. 2013/0301888, 2013/0316914, and 2014/0045175, as well as U.S. Pat. No. 9,017,973, all of which are incorporated herein by reference.

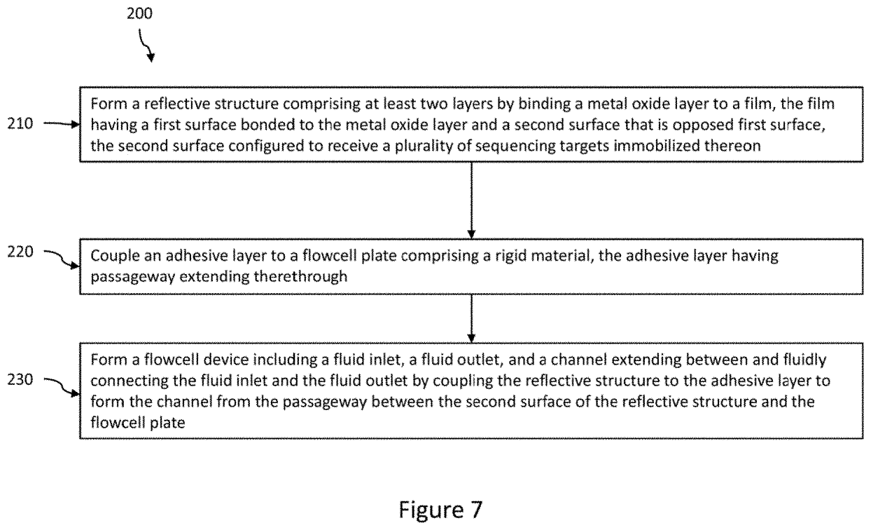

FIG. 7 depicts an exemplary embodiment of a method 200 for manufacturing a flowcell device for sequencing by synthesis instruments. As a general overview, method 200 includes the steps of: forming a reflective structure comprising at least two layers, coupling an adhesive layer to a flowcell plate, and forming a flowcell device by coupling the reflective structure to the adhesive layer.

In step 210, a reflective structure comprising at least two layers is formed by binding and/or coupling a metal oxide layer to a film. The film has a first surface bonded to the at least one metal oxide layer and a second surface opposed the first surface that is configured to receive a plurality of sequencing targets immobilized thereon. The film may be bonded to the metal oxide layer by way of chemical interaction, mechanical interactions, electrostatic interaction, or the like. The layers of the reflective structure may be bonded together by way of chemical interactions, electrostatic interactions, or any other known means for producing stable structures having two or more layers on a nanometer scale. For example, the metal oxide layer may be bonded and/or disposed onto the film or a substrate by sputtering and/or by atomic layer deposition.

Sputtering may be performed using a high vacuum technique or other suitable method to fabricate the reflective structure. The structure of the material forming the layer(s) of the reflective structure can be influenced by the substrate temperature, the carrier gas flow, and the sputtering power. Due to the formation of sub-structures and regions of low crystallinity, a suitable barrier layer from sputtering may be required to have thickness of at least 20 nm, preferably at least 50 nm, or preferably at least 100 nm.

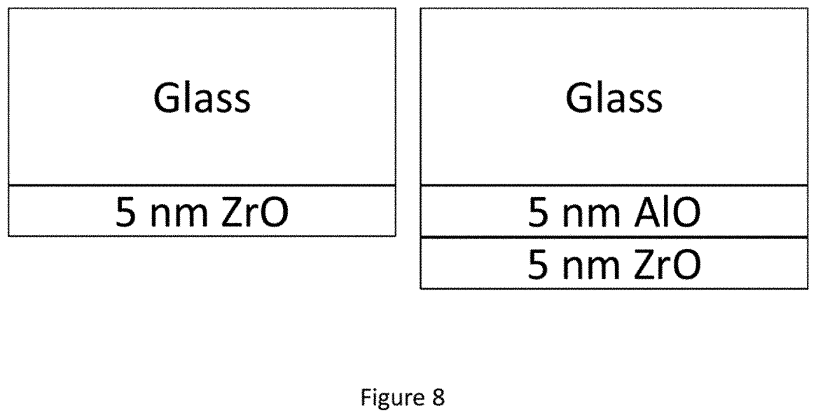

Atomic layer deposition is a self-limiting surface reaction of gases that leads to the formation of mono-layers of target materials. Materials having a thickness in the range of nanometers may generally be produced by subsequent cycles of atomic layer deposition. Through atomic layer deposition fabrication techniques, fully amorphous layers with a thickness of 5 nm and suitable barrier properties can be obtained. Barrier layers may be realized by sandwiching metal oxide layers of differing materials (thereby, enabling differing material properties to be obtained) as shown in FIG. 8. Additionally and/or alternatively, it is possible to use atomic layer deposition techniques to decouple the barrier properties of a metal oxide layer 134 from the surface properties of a substrate and/or film 140 (e.g., for the attachment of DNA retaining materials).

In step 220, an adhesive layer is coupled to a flowcell plate comprising a rigid material. The adhesive layer has a passageway extending therethrough, e.g., from a first surface of the adhesive layer to a second opposed surface of the adhesive layer. The adhesive layer may be coupled to the flowcell plate using techniques known to one of skill in the art. Preferably, the adhesive layer is sealed to the flowcell plate such that fluid reagents flowing through the passageway do not exit the flowcell device except through the fluid outlet—thereby restricting and/or preventing flow through a gap between the adhesive layer and the flowcell plate.

In step 230, a flowcell device is formed to include a fluid inlet, a fluid outlet, and a channel extending between and fluidly connecting the fluid inlet and the fluid outlet by coupling the reflective structure to the adhesive layer to form the channel from the passageway between the second surface of the reflective structure and the flowcell plate. The reflective structure may be coupled to the adhesive layer using techniques similar to or different from the techniques used to couple the adhesive layer to the flowcell plate. For example, the reflective structure may be coupled to the adhesive layer using any technique that does not interfere with the properties and characteristics of the film or the plurality of sequencing targets thereon. Preferably, the plurality of sequencing targets on the film are positioned within the channel upon the flowcell device being formed. For example, coupling the reflective structure to the adhesive layer may position the plurality of sequencing targets with respect to the channel such that the plurality of sequencing targets may contact the fluid reagents flowing therethrough. In one embodiment, the film is in fluid connection with the channel upon the flowcell device being formed.

According to another embodiment of the invention, a barrier layer may be employed between film 140 and metal oxide layer 134 of reflective structure 130. The barrier layer may, preferably, be compatible with film 140 and have high barrier properties against moisture, high temperature and chemical resistance, and good optical properties. Film 140 may be utilized to bind and retain the barrier layer to the metal oxide layer of reflective structure 130 by, e.g., applying the barrier polymer and subsequently treating film 140 with ultraviolet (“UV”) light exposure to covalently attach the barrier layer and/or film 140 to the metal oxide layer of reflective structure 130. Alternatively and/or additionally, the barrier layer may be coupled to the metal oxide layer of reflective structure 130 by a layer-by-layer technique. In accordance with a layer-by-layer technique, film 140 may be positively charged and a second anionic component may subsequently added, thereby creating a cationic-anionic-cationic interface, which is stabilized due to strong electrostatic interaction between the layers before curing to create a cross-linked structure.

### EXAMPLES

The following examples are non-limiting embodiments of the present invention, included herein to demonstrate the advantageous utility obtained from aspects of the present invention.

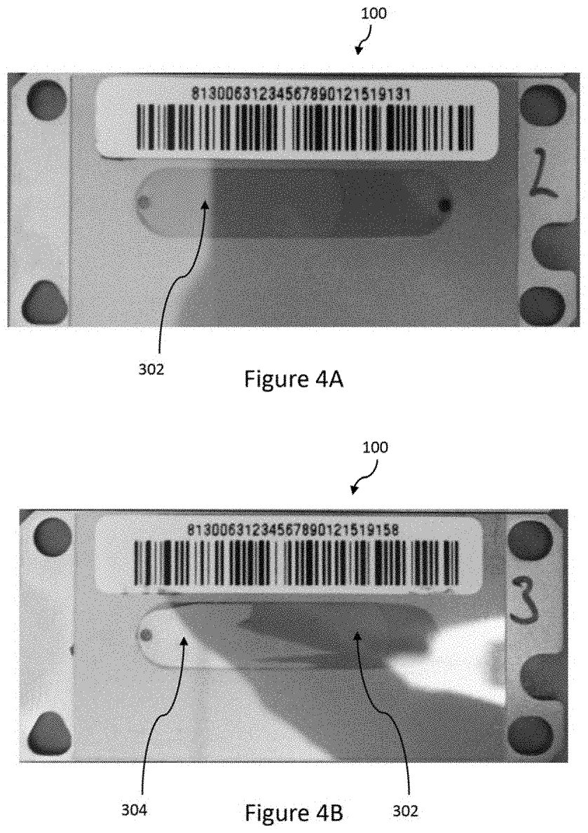

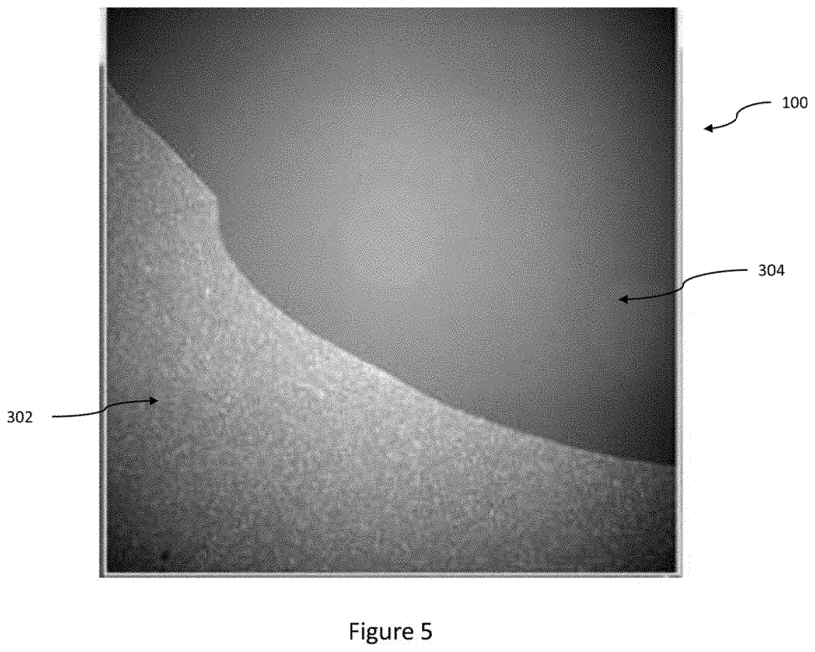

During the development of the flowcell device, the present inventors discovered that certain chemical species, or combination of chemical components, lead to a pronounced loss of sequencing targets during sequencing runs using a flowcell device having a reflective structure formed by sputtering. This effect was discovered to occur, particularly, with chemical compositions relating to gallic acid (hereafter “GA”), hydrogen peroxide, and dimercaptopropanesulfonic acid (hereafter “DMPS”). These effects were also recognized to occur with other chemical compositions. A typical failure resulting from a significant loss of sequencing targets induced by chemistry containing GA is shown in FIGS. 4B and 5. FIG. 4B illustrates that the loss of sequencing targets from damaged region 304 of the reflective structure may be noticeable with the naked eye. FIG. 4A illustrates a flowcell that does not have a significant loss of sequencing targets. FIG. 5 is a microscopic image of a reflective structure having a damaged portion 304 and a non-damaged portion 302.

The present inventors investigated the loss of sequencing targets by analyzing the inner surface of the flowcell device and determined that there was a loss of the upper-most silicon-oxide layer of the optical mirror structure through chemistry induced etching. Due to the chemical etching of the silicon oxide layer, the inventors determined that the film (e.g., an AZIGRIP4 coating) was freely floating and could be removed by the fluidic flow in the channel area. This type of problem is termed herein as delamination of film, but the invention is not limited to any specific mechanics of the process by which the film separates.

The present inventors further recognized that the delamination of film can occur with devices, other than flowcell devices, whereby damage to the bulk of glass induces the delamination of film. This determination indicates that the delamination of film is not related to the special case of sputtered silicon oxide layers in flowcell devices, but also applies more generally to devices employing glass and glass-like materials with certain chemical compounds. Due to the presence of native oxide layers on silicon wafers (through handling of wafers in ambient conditions), this finding is of relevance for disposables based on silicon wafer technology.

The inventors have developed a more robust flowcell device by including barrier properties into the layout of the reflective structure itself. In one non-limiting example, the barrier properties were included into the reflective structure itself by including a penultimate layer of 100 nm Niobium-pentoxide (Nb₂O₅). The inventors also determined that the thickness of the metal oxide layer can be important for its barrier properties. For layers produced by a sputtering processes (which was used in this example to obtain the optical mirror structure), a minimum thickness of 20 nm was considered to be a minimum value for obtaining a closed layer without pinholes or defects for suitable barrier properties. A thickness of 100 nm was found to be an optimum thickness for the transmissive properties of the complete reflective structure (lowest possible loss of light in the spectral region 400-700 nm due to reflection from the structure). The infrared reflection properties necessary for the autofocus system were not affected by the change between reflective structures having a penultimate layer of metal oxide and the optical mirror structures having a terminal layer of silicon oxide.

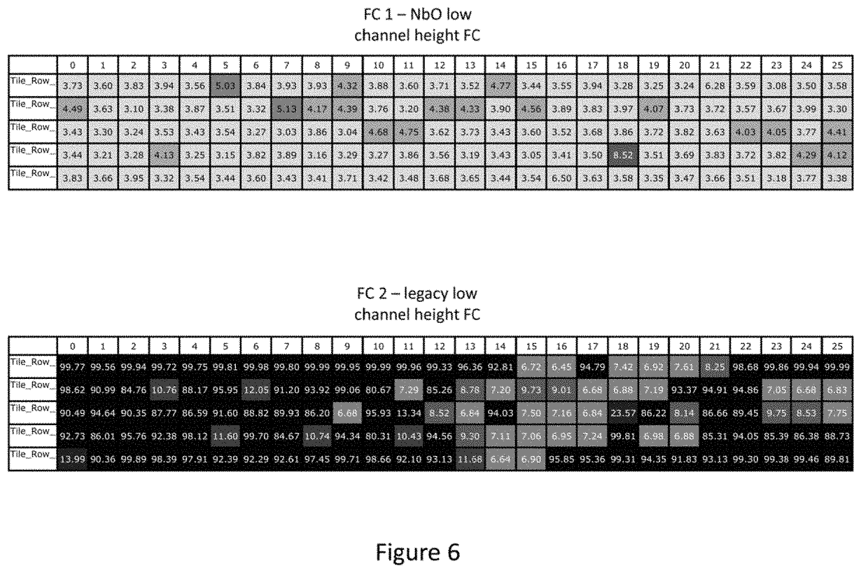

A flowcell having a reflective structure with a penultimate layer comprising niobium oxide was compared to a flowcell having a reflective structure with a penultimate layer comprising silicon oxide to determine the benefits of utilizing a metal oxide layer as the penultimate layer of the reflective structure. Both samples were processed with the same sequencing instrument, using the same chemistry, and by employing sequencing targets originating from the same pool. The results of the measurements are shown as a heat map in FIG. 6. The numbers in the heat map indicate the percent of beads lost during the process, which has been coded from lighter shading (low loss) to darker shading (high loss) in FIG. 6.

From the results of FIG. 6, it is clear that the flowcell device having a reflective structure with a penultimate layer of silicon oxide had been severely damaged by the SBS chemistry (containing DMPS and hydrogen peroxide), which lead to high losses of sequencing targets due to delamination and film damage. The flowcell having an reflective structure with a penultimate layer comprised of niobium oxide did not show any damage related to delamination of film.

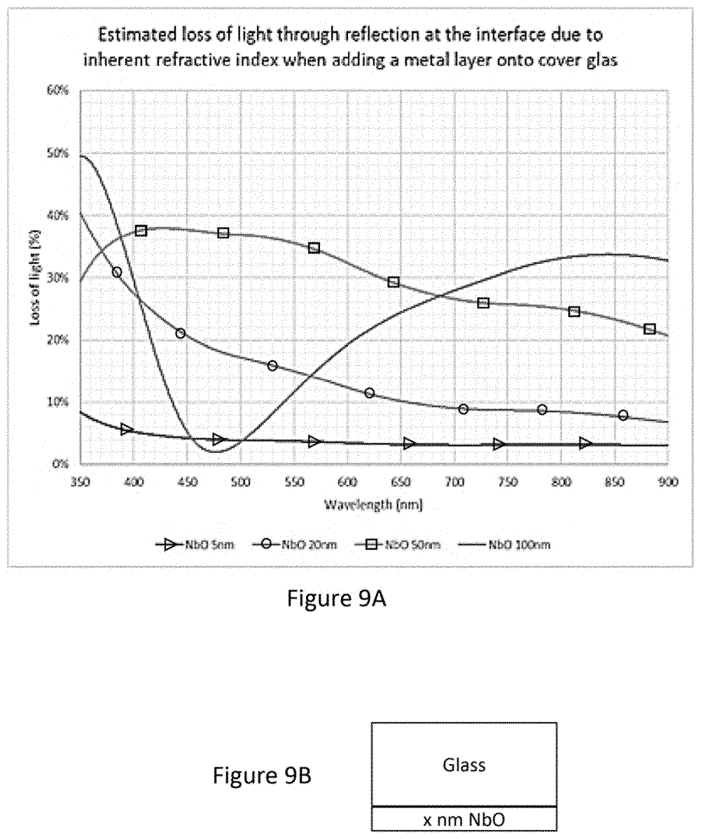

The inventors also investigated the effect of the high refractive index of metal oxides by testing a substrate of glass with a metal oxide layer at various thicknesses (see FIG. 9B). FIG. 9A shows a simulated plot on the reflection for a glass coated with various thicknesses of niobium oxide. The simulation was based on interpolation of single refractive index data points for niobium-pentoxide. FIG. 9A implies that a very thin layer of Nb₂O₅ is preferable in order to keep optical losses through reflection at a minimum. However, for the metal oxide layer to have a sufficient barrier effect for the sequencing chemistry, the actual structure of the reflective structure needs to be considered. Barrier properties are more distinct for purely amorphous layers over low crystalline content and poly-crystalline materials. The structure may also be influenced by the fabrication technique utilized for producing those layers.

Although the invention is illustrated and described herein with reference to specific embodiments, the invention is not intended to be limited to the details shown. Rather, various modifications may be made in the details within the scope and range of equivalents of the claims and without departing from the invention. Numerous variations, changes and substitutions will occur to those skilled in the art without departing from the spirit of the invention. Accordingly, it is intended that the appended claims cover all such variations as fall within the spirit and scope of the invention. Additional descriptions of flowcells are found in U.S. Pat. Nos. 8,481,259, 8,940,481, and 9,146,248, and U.S. Patent Application Publication Nos. 2009/0298131 and 2014/0267669, all of which are incorporated herein by reference.

1. A flowcell device for a sequencing by synthesis instrument, the flowcell device comprising:
a fluid inlet configured to receive one or more liquid reagents;
a fluid outlet configured to pass the one or more liquid reagents; and
a channel extending between and fluidly connecting the fluid inlet and the fluid outlet, wherein at least a portion of the channel comprises a reflective structure configured to retain a plurality of sequencing targets thereon, the reflective structure having a metal oxide layer and a film having a first surface and a second surface opposed the first surface, the first surface of the film disposed on the at least one metal oxide layer and the second surface of the film configured to receive a plurality of sequencing targets immobilized thereon.

2. The flowcell device of claim 1, wherein the reflective structure is configured to reflect near-infrared light waves.

3. The flowcell device of claim 2, wherein the reflective structure is arranged such that the film is a terminal layer of the reflective structure.

4. The flowcell device of claim 3, wherein the reflective structure is arranged such a penultimate layer of the reflective structure is a metal oxide layer.

5. The flowcell device of claim 3, wherein the film is configured to hold the plurality of sequencing targets immobilized on the film in fluid connection with the channel.

6. The flowcell device of claim 1, wherein the entirety of the second surface of the film is disposed on the metal oxide.

7. The flowcell device of claim 1, wherein the metal oxide layer comprises niobium oxide.

8. The flowcell device of claim 1, wherein the metal oxide layer comprises zirconium oxide.

9. The flowcell device of claim 1, wherein the metal oxide layer comprises titanium oxide.

10. The flowcell device of claim 1, wherein the metal oxide layer comprises hafnium oxide.

11. The flowcell device of claim 1, further comprising:
a flowcell plate comprising a rigid material; and
an adhesive layer having a passageway extending therethrough, the adhesive layer disposed between the flowcell plate and the reflective structure, such that the passageway forms the channel between the second surface of the film of the reflective structure and the flowcell plate.

12. The flowcell device of claim 11, wherein the film is configured such that the plurality of sequencing targets immobilized on the film are positioned within the channel.

13. A method of manufacturing flowcell devices configured for sequencing by synthesis, the method comprising:
forming a reflective structure comprising at least two layers by binding a metal oxide layer to a film, the film having a first surface bonded to the metal oxide layer and a second surface that is opposed first surface, the second surface configured to receive a plurality of sequencing targets immobilized thereon;
coupling an adhesive layer to a flowcell plate comprising a rigid material, the adhesive layer having passageway extending therethrough; and
forming a flowcell device including a fluid inlet, a fluid outlet, and a channel extending between and fluidly connecting the fluid inlet and the fluid outlet by coupling the reflective structure to the adhesive layer to form the channel from the passageway between the second surface of the film of the reflective structure and the flowcell plate.

14. The method of claim 13, wherein the plurality of sequencing targets immobilized on the film are positioned within the channel.

15. The method of claim 13, wherein the plurality of sequencing targets immobilized on the film are in fluid connection with the channel.

16. The method of claim 13, wherein the metal oxide is formed by sputtering.

17. The method of claim 13, wherein the metal oxide is formed by atomic layer deposition.
